(19) United States
(12) Patent Application Publication
(10) Pub. No.: US 2018/0199836 A1
(43) Pub. Date: Jul. 19, 2018

(54) BIOLOGICAL INFORMATION DETECTION DEVICE, BIOLOGICAL INFORMATION DETECTION METHOD, AND BIOLOGICAL INFORMATION DETECTION SYSTEM

(71) Applicant: PANASONIC INTELLECTUAL PROPERTY MANAGEMENT CO., LTD., Osaka (JP)

(72) Inventors: Masao HAMADA, Fukuoka (JP); Tsuyoshi NAKAMURA, Fukuoka (JP); Tadanori TEZUKA, Fukuoka (JP)

(73) Assignee: PANASONIC INTELLECTUAL PROPERTY MANAGEMENT CO., LTD., Osaka (JP)

(21) Appl. No.: 15/867,173

(22) Filed: Jan. 10, 2018

(30) Foreign Application Priority Data
Jan. 16, 2017 (JP) 2017-005175

(51) Int. Cl. A61B 5/024; A61B 5/00

(52) U.S. Cl. CPC A61B 5/02416; A61B 2576/00; A61B 5/7485

(57) ABSTRACT

A biological information detection device includes an image input that inputs image data captured by an observed person, an image region divider that divides the image data into a plurality of regions, a biological determiner that determines presence or absence of detection of biological information to be estimated as a pulse wave of a person in each of the divided regions, a biological information and position storage that obtains detected biological information and the position information of the region in which the biological information is detected, and stores the biological information and the position information.

### BACKGROUND

### 1. Technical Field

The present disclosure relates to a biological information detection device, a biological information detection method, and a biological information detection system capable of detecting biological information of a person.

### 2. Description of the Related Art

In the related art, for example, a vital sensing technology for estimating biological information of a person is not limited to the fields of home health care and health management. It is expected that the technology is applied to applications such as detection of drowsiness during driving, acquisition of a psychological state of a user during the game, and detection of abnormal persons in a monitoring system. Currently, devices that sense the biological information are mainly used in contact with the body of a person, and it is necessary for a user to wear the devices such that the range of application is limited.

For this reason, as one approach to non-contact sensing, a technology for estimating a pulse rate as an example of the biological information from an image obtained by camera capturing has been proposed. By using the technology, it is possible to sense the biological information without making user conscious of himself, and it is expected that the range of application can be expanded. For example, it is possible to detect a suspicious person whose pulse variation increases due to stress while capturing with the monitoring camera. In addition, it is also a great advantage that it is possible to simultaneously sense multiple people captured by one camera. Compared with a contact type, it is not necessary to prepare individual devices for each user, and it is possible to reduce troublesome that it is attached to the body.

Here, as a prior art for observing (detecting) a biological state of a person, for example, a biological state observation system shown in International Publication No 2014/030439 is known. The biological state observation system inputs an image including at least one observed person and configured with a plurality of temporally consecutive frames, outputs information of a predetermined region by detecting a predetermined region in an image, detects biological information of the observed person included in the image from a predetermined region in the image, compares a predetermined biological parameter used for determining abnormality in the state of a biological body and data of biological information or change of the data, and detects the abnormality occurring in a biological state of the observed person. With this, the biological state observation system can observe the biological information of the observed person and detect the abnormality of the biological state without giving an unnatural feeling or discomfort to the observed person.

In International Publication No 2014/030439 described above, by using a known face detection technology, the face region of the observed person is detected, a region of the skin of the face region is set as a predetermined region in an image, and the biological information is detected from the predetermined region. However, in a configuration International Publication No 2014/030439, there is a problem that the biological information such as the pulse rate cannot be detected in a state where face detection cannot be normally performed such as the face of the observed person turns sideways.

### SUMMARY

An object of the present disclosure devised in view of the above-described circumstances in the related art is to provide a biological information detection device, a biological information detection method, and a biological information detection system which accurately detect biological information of an observed person even in a case where face detection of the observed person cannot be performed.

According to the present disclosure, there is provided a biological information detection device including an image input that inputs image data captured by an observed person; an image region divider that divides the image data into a plurality of regions; a biological determiner that determines presence or absence of detection of biological information to be estimated as a pulse wave of a person in each of the regions; a biological information obtainer that obtains the detected biological information; and a position information obtainer that obtains position information of a region in which the biological information is detected.

In addition, according to the present disclosure, there is provided a biological information detection method in a biological information detection device, the method including receiving image data captured by an observed person; dividing the image data into a plurality of regions; determining presence or absence of detection of biological information to be estimated as a pulse wave of a person in each of the regions; obtaining the detected biological information; and obtaining position information of a region in which the biological information is detected.

In addition, according to the present disclosure, there is provided a biological information detection system including a biological information detection device that detects biological information based on image data captured by an observed person and a biological information processing device that processes the biological information obtained by the biological information detection device, in which the biological information detection device receives the image data captured by the observed person, divides the image data into a plurality of regions, determines presence or absence of detection of the biological information to be estimated as a pulse wave of a person in each of the regions, obtains the detected biological information, obtains position information of a region in which the biological information is detected; and outputs the biological information and the position information to the biological information processing device, and the biological information processing device receives the biological information and the position information, detects abnormality of the received biological information, detects abnormality of the received position information, and notifies the abnormality in a case where an abnormal state of at least one of the biological information and the position information is detected.

According to the present disclosure, even in a case where the face detection of the observed person cannot be performed, it is possible to accurately detect the biological information of the observed person.

### BRIEF DESCRIPTION OF THE DRAWINGS

FIG. 1 is an explanatory diagram showing an example an overview of a biological information detection system of a present embodiment;

FIG. 2 is a block diagram showing a functional configuration of the biological information detection system of a first embodiment;

FIG. 3A is a diagram schematically showing an example of a relationship between the shrinkage of person heart and the absorbed amount of light in a blood vessel;

FIG. 3B is a diagram showing an example of time series change of the light intensity;

FIG. 4 is a diagram showing an example of an absorption rate for each wavelength of light in hemoglobin;

FIG. 5 is a diagram showing an example of a captured image obtained by capturing an inside of a room including a watching target person;

FIG. 6 is a diagram for explaining a biological information detection process in the first embodiment;

FIG. 7 is a flowchart showing a procedure of the biological information detection process of the first embodiment;

FIG. 8 is a flowchart showing of an abnormal state detection process in the present embodiment;

FIG. 9 is a block diagram showing a functional configuration of the biological information detection system of a second embodiment;

FIG. 10 is a diagram for explaining a biological information detection process of the second embodiment;

FIG. 11 is a flowchart showing a procedure of the biological information detection process of the second embodiment;

FIG. 12 is a block diagram showing a functional configuration of a biological information detection system of a third embodiment;

FIG. 13 is a diagram for explaining a biological information detection process in a first example of the third embodiment;

FIG. 14 is a diagram for explaining a re-detection process in a case where biological information cannot be detected;

FIG. 15 is a flowchart showing a procedure of the biological information detection process of the first example of the third embodiment;

FIG. 16 is a diagram for explaining a biological information detection process in a second example of the third embodiment; and

FIG. 17 is a flowchart showing a procedure of the biological information detection process in the second example of the third embodiment.

### DETAILED DESCRIPTION

Hereinafter, with reference to appropriate drawings, an embodiment (hereinafter, referred to as “present embodiment”) in which a biological information detection device, a biological information detection method, and a biological information detection system according to the present disclosure are specifically disclosed will be described below in detail. However, more detailed explanation than necessary may be omitted. For example, a detailed explanation of well-known matters and redundant explanation for substantially the same configuration may be omitted. This is to avoid unnecessary redundancy of the following description and to facilitate understanding by those skilled in the art. The accompanying drawings and the following description are provided to enable those skilled in the art to fully understand the present disclosure and are not intended to limit the subject matter recited in the claims by these.

For example, the biological information detection device of the present embodiment detects the pulse rate of a person in a non-contact manner by using image data captured from an object (for example, referred to as person hereinafter), as an example of the biological information without using a dedicated pulse rate measuring device of a contact type. Hereinafter, as an example of the biological information detection device and the biological information detection system, a configuration example in which a space in a room where a watching target person is present is captured, a biological body is detected from the captured image data, and the biological information is obtained is indicated.

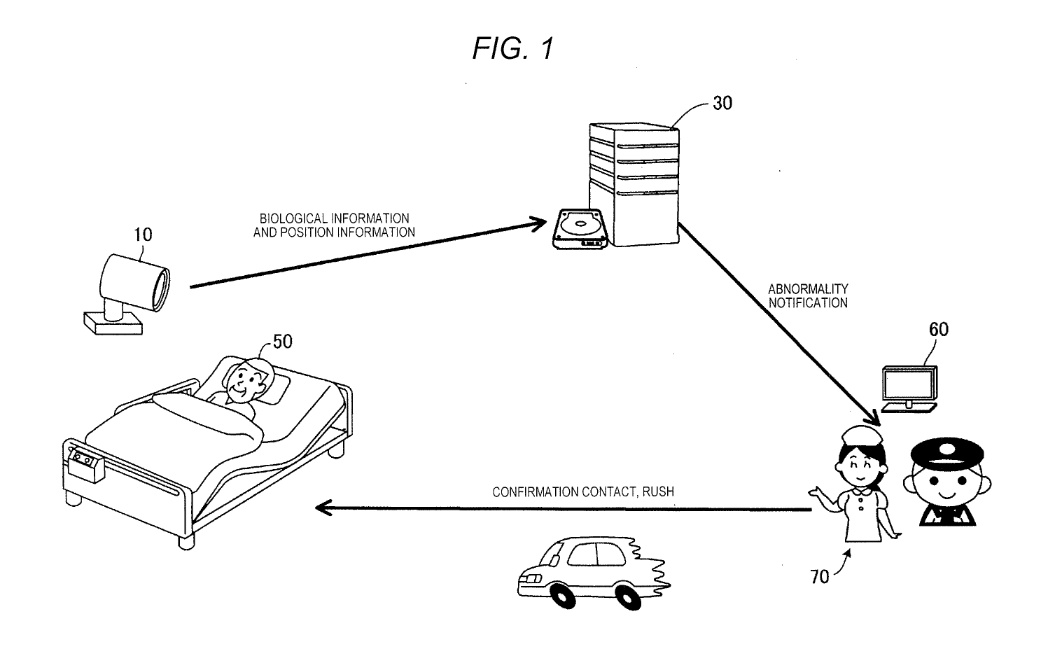

FIG. 1 is an explanatory diagram showing an example of an overview of the biological information detection system of the present embodiment. The biological information detection system is configured with camera 10 that functions as an example of the biological information detection device, and server 30 that functions as an example of the biological information processing device to be processed by obtaining the image data of a monitoring target, the biological information, and position information of the observed person including an observed person obtained by camera 10. Here, as an example of the observed person, the biological information detection system where a room in which watching target person 50 such as senior citizens is present is captured and detection of the biological information of watching target person 50, detection of an abnormal state, and the like are performed, is exemplified.

Camera 10 is an imaging device such as the monitoring camera for capturing indoor, installed, for example, on the ceiling of a room, and captures an inside of a room including watching target person 50 downward from the vicinity of the ceiling toward the floor. Camera 10 includes a capture including a lens and an imaging element, an information processor including a processor and a memory, and a communicator including a communication circuit. Camera 10 detects the biological information of watching target person 50 and the position information, and transmits the obtained biological information of watching target person 50 and the position information to server 30 based on the captured image data of the inside of the room. In addition, the image data including watching target person 50 captured by camera 10 may be transmitted to server 30. Camera 10 and server 30 are connected to each other through a network such as a wired network and a wireless network, or another communication line, and can transmit and receive data by performing communication therebetween.

Server 30 includes a computer including a processor and a memory and a storage for recording various pieces of data. Server 30 stores by recording the image data obtained by camera 10, the biological information, and the position information, and detects abnormality of the biological information and the position information. In a case where the abnormal state is detected in at least one of the biological information and the position information, as an example of another device, server 30 transmits abnormality notification to terminal 60 used by watcher 70 watching over watching target person 50. For example, watcher 70 is a nurse nursing watching target person 50, a provider providing watch service, a security guard who guards a building where watching target person 50 resides, a service customer such as a close relative who receives the watch service, and the like. Terminal 60 is configured with a communication terminal such as a personal computer and a smartphone, a mobile phone, and an alarm such as a speaker, a lamp, and a buzzer, receives data and signals in association with the abnormality notification by being connected to server 30, and notifies watcher 70 that the abnormality occurs. Terminal 60 is disposed at a nurse center in which the nurse resides, a monitoring center of the watch service provider, a customer base of the watch service, and the like. When the occurrence of an abnormality is recognized, watcher 70 performs correspondence behaviors at the time of the occurrence of an abnormality such as confirmation contact with respect to watching target person 50 and rushes to the site. It is possible to transmit the biological information from server 30 to terminal 60 for each predetermined time or at an arbitrary timing, and it is possible to monitor the biological information by displaying the biological information of watching target person 50 on terminal 60 regardless of the presence or absence of occurrence of an abnormality.

### First Embodiment

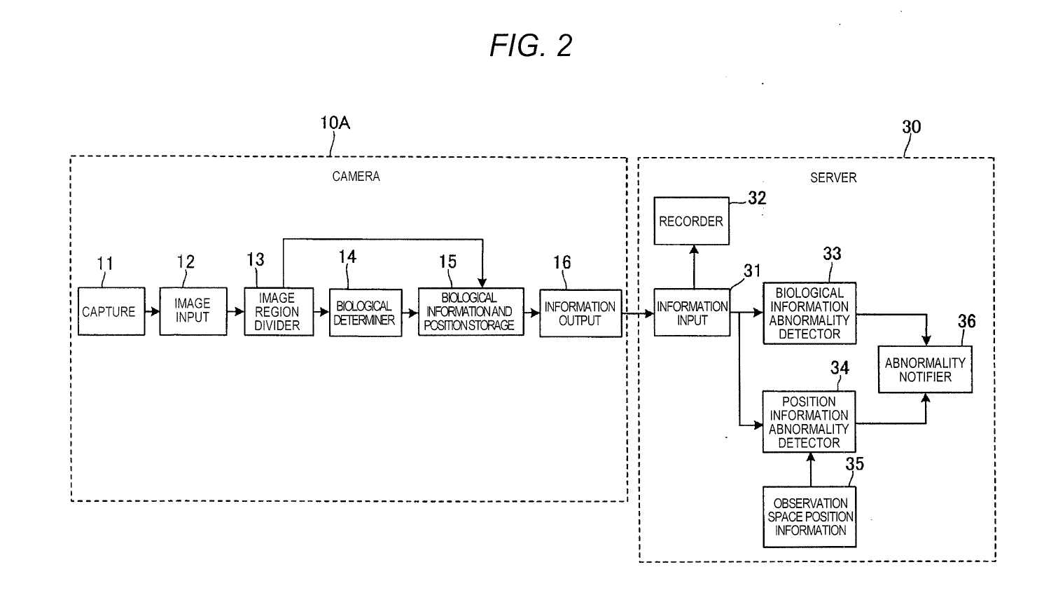

FIG. 2 is a block diagram showing a functional configuration of the biological information detection system of the first embodiment. In the first embodiment, camera 10A includes capture 11, image input 12, image region divider 13, biological determiner 14, biological information and position storage 15, and information output 16. Capture 11 includes a solid-state imaging element such as an imaging lens, a charge coupled device type image sensor (CCD), or a complementary metal oxide semiconductor (CMOS) type image sensor. Capture 11 outputs the image data of an object obtained by capturing the inside of a room including watching target person 50.

Each of functions of image input 12, image region divider 13, biological determiner 14, biological information and position storage 15 is realized by the information processor including a processor and a memory. Camera 10A may be configured with only capture 11 and the biological information detection device may be configured with another configuration different from that of a camera not including the capture. For example, in this case, the camera and the biological information detection device are connected through a network such as the wired network, the wireless network, and another communication line.

Image input 12 successively inputs (obtains) the frames of the image data captured by capture 11 at a predetermined frame rate, and outputs the input frame to image region divider 13. In a case where the camera and the biological information detection device are separately configured, image input 12 successively inputs the frames of the image data input from capture 11.

Image region divider 13 divides the image data of the inside of a room obtained by image input 12 into a plurality of regions. For example, image region divider 13 divides the image data into grid type regions of a plurality of regions by dividing the image data into the plurality of regions in the vertical direction and the horizontal direction of the image. Image region divider 13 outputs the position information of the region of each divided region to biological information and position storage 15.

Biological determiner 14 determines whether or not the biological information to be estimated as a pulse wave of a person is detected with respect to the image data of each divided region, and determines whether or not the biological body is present in the region of the captured image. For example, biological determiner 14 may adopt a configuration having a filter, a waveform verifier, and a pulse estimator in order to perform an information process to which a biological information estimation technology disclosed in PTL 1 below is applied.

PTL 1: Japanese Patent Unexamined Publication No. 2016-077539

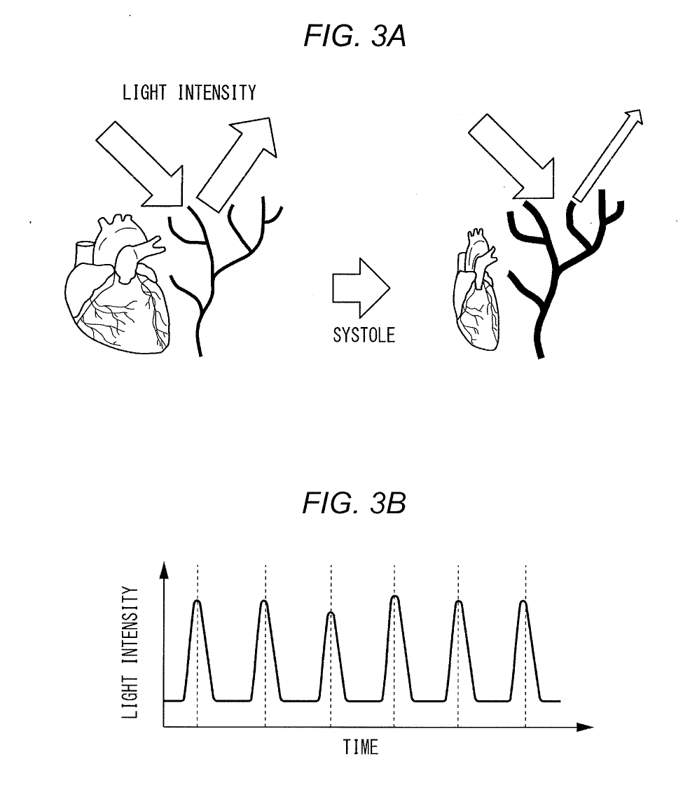

Here, an estimation principle of a pulse rate in the biological information detection device of the present embodiment will be described with reference to FIG. 3A and FIG. 3B. FIG. 3A is a diagram schematically showing an example of a relationship between the shrinkage of the person heart and the absorbed amount of light in a blood vessel. FIG. 3B is a diagram showing an example of a time series change of the light intensity.

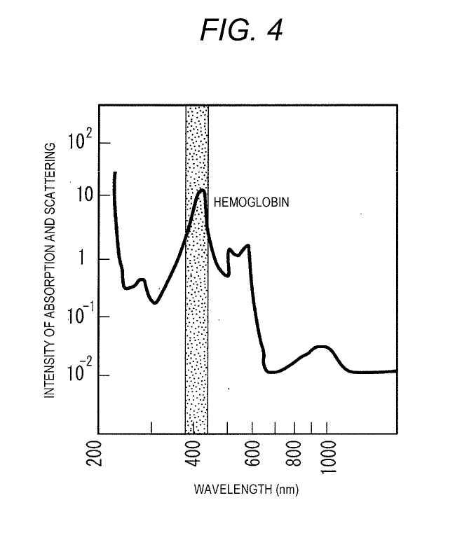

In FIG. 3A, change in a volume of the blood vessel synchronously with the shrinkage (systole) of the person heart is shown. When the volume of the blood vessel increases according to the shrinkage of heart, since the absorbed amount of light (for example, light in specific wavelength region shown in FIG. 4) increases, the light intensity also decreases (see FIG. 3B). The pulse wave shows the motion of a wave when the pressure change in the blood vessel generated when blood is pushed out to the aorta due to the shrinkage of the heart is transmitted in the peripheral direction.

In FIG. 3B, the horizontal axis represents time, and the vertical axis represents the intensity of a signal (photoelectric pulse wave) obtained by the change of the absorbed amount of light. That is, in FIG. 3B, when a peak appears, since the absorbed amount of light is small, the volume of the blood vessel does not increase, and when the minimum value appears, since the absorbed amount of light increases, the volume of the blood vessel increases. Although it is not perfectly synchronized with a distance between the heart and the peripheral part, there is some delay. However, the shrinkage of the heart and the change in the intensity of the photoelectric pulse wave are varied by basically synchronizing with each other.

FIG. 4 is a diagram showing an example of an absorption rate for each wavelength of light in hemoglobin. In FIG. 4, for example, it is shown that the hemoglobin (blood) easily absorbs a wavelength of 400 nm (that is green). In each of the following embodiments, description will be described by using the fact that the absorption rate of the component of green light is high. However, in addition to this, it may be described by using the fact that the reflectance of the component of red light (for example, wavelength exceeding 1,000 nm) is high.

In biological determiner 14, the filter is configured with a bandpass filter having a predetermined filter coefficient. For example, the filter removes a noise signal included at the time of capturing by capture 11 by averaging signals (pixel values) in a predetermined range of input image data. By this averaging, in a case where a person is present in the region of the captured image, the pulse wave of a person can be extracted. However, because of a high possibility that residual components of a body motion component and noises are still included, the filter uses a set filter coefficient to cut frequency components other than the fundamental frequency of the pulse wave. The output of the filter is input to the waveform verifier and the pulse estimator.

For example, the filter coefficient of the filter is set in advance to pass signals of 30 to 120 bpm through the filter. Generally, an adult's pulse at rest is between 60 and 80 bpm, but here, a passing target range of the filter is set to 30 to 120 bpm in consideration of the influence of watching target person 50 at the time of bradycardia or tachycardia or at the time of exercise and tension.

The waveform verifier determines whether or not there is a section of a signal that satisfies a predetermined condition corresponding to a noise signal in an output signal to be input corresponding to at least one cycle of the filter, in order to detect a section in which the output signal corresponding to at least one cycle of the filter is input and the noise signal cannot be cut through the filter. The waveform verifier excludes the section of the output signal in which it is determined that the predetermined condition is satisfied as an invalid section. For example, the waveform verifier excludes an extremely large signal and an extremely small signal as compared to a predetermined value (for example, zero). In a case where it is determined that there is the section of the signal satisfying the predetermined condition, the waveform verifier outputs at least one cycle signal in which a corresponding signal section is excluded, to the pulse estimator. Meanwhile, in a case where it is determined that there is no section of the signal satisfying the predetermined condition, the waveform verifier outputs the output signal for at least one cycle from the filter to the pulse estimator as it is. A process of the waveform verifier uses a knowledge that an amplitude of the pulse wave of a person changes gently in a constant width, and the signal satisfying the predetermined condition is highly likely to be disturbance noise.

The pulse estimator calculates the pulse rate (pulse rate) of a person based on input intervals of frames of the image data by using the output signal for at least one cycle of the filter or the output signal for at least one cycle of the waveform verifier.

In a case where the pulse rate is normally calculated in the pulse estimator, biological determiner 14 determines that a person (that is, watching target person 50) is present in the region of the captured image. Meanwhile, in a case where the pulse rate is not normally calculated, biological determiner 14 determines that the person is not present in a corresponding region of a captured image. Biological determiner 14 determines the presence or absence of the biological body in each region of the divided image data. In a case where it is determined that there is a person, biological determiner 14 outputs the biological information including the pulse rate of watching target person 50 detected from the captured image in the region to biological information and position storage 15. At this time, biological determiner 14 determines that the biological body is present, and a position of watching target person 50 in the inside of a room is detected based on a position of the region in an image in which the biological information is detected.

Biological information and position storage 15 is an example of a configuration for realizing functions of a biological information obtainer and a position information obtainer, configured with a memory, and stores the position information of a region of the image data output by image region divider 13 and the biological information of watching target person 50 output by biological determiner 14. Biological information and position storage 15 stores the position information of the region in which the biological information is detected as the position information of watching target person 50 by corresponding the position information of each region and the region in which the biological information is detected. For example, information output 16 is configured with a communication circuit, an output interface, and the like, outputs the biological information and the position information stored in biological information and position storage 15, and transmits the output information to server 30.

Server 30 includes information input 31, recorder 32, biological information abnormality detector 33, position information abnormality detector 34, and abnormality notifier 36. For example, information input 31 is configured with a communication circuit, an input and output interface, and the like, and inputs the biological information and the position information output from information output 16. For example, recorder 32 is configured with a storage such as a semiconductor memory and a hard disk device, and records and stores the biological information and the position information obtained by biological determiner 14. In addition, recorder 32 records in advance observation space position information 35 indicating a position of an element present in the inside of a room such as a bed, furniture, and fixtures included in the image data of the monitoring target captured by capture 11. Recorder 32 may record and store the image data obtained by capture 11.

Each function of biological information abnormality detector 33 and position information abnormality detector 34 is realized by the information processor including a processor and a memory. Biological information abnormality detector 33 monitors the obtained biological information of watching target person 50, and detects the abnormal state of the biological information such as sharp increase, decrease, and stop of the pulse rate. Position information abnormality detector 34 monitors the obtained position information of watching target person 50 and detects the abnormal state of the position information such as determining that watching target person is fallen from the bed in a case where, for example, watching target person 50 is present at a position other than the bed for a predetermined time or more by being compared with observation space position information 35. In addition, in a case where the position information is not detected for a predetermined time or more such as watching target person 50 leaves the room, the abnormal state of the position information may be determined.

For example, abnormality notifier 36 is configured with a communication circuit and the output interface, outputs the abnormality notification in a case where the abnormal state is detected in the biological information or the position information, and transmits the abnormality notification to terminal 60 used for watcher 70. For example, the abnormality notification can be performed by using various elements such as a notification signal, character information, image information, and audio information indicating the occurrence of an abnormality.

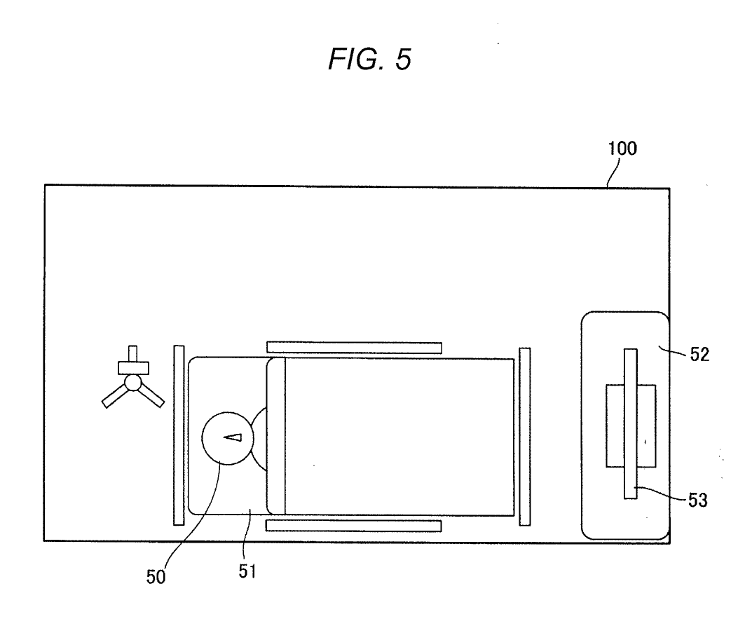

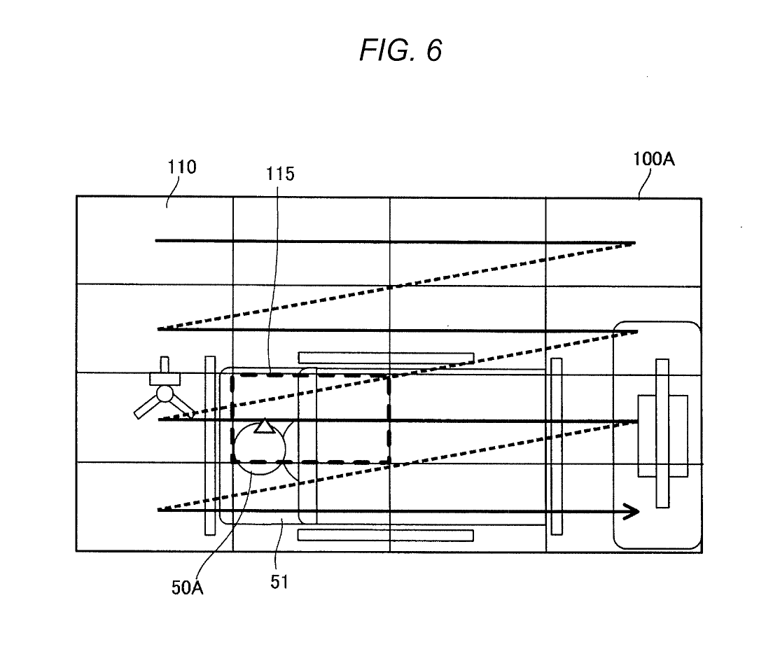

Next, each process in the detection of the biological information and detection of the abnormal state in the first embodiment will be described. FIG. 5 is a diagram showing an example of the captured image obtained by capturing the inside of a room including watching target person 50. FIG. 6 is a diagram for explaining the biological information detection process in the first embodiment.

Captured image 100 obtained by capture 11 includes watching target person 50, bed 51, furniture 52, TV 53, and the like as an image of the monitoring target of the inside of a room. Here, it is assumed that watching target person 50 is sleeping on bed 51. Biological determiner 14 detects the biological information from the image information of a face portion of watching target person 50 in captured image 100. In an example of FIG. 5, since watching target person 50 directly faces upward and the front of a face is captured, it is possible to extract a face region by performing a known face detection process and to obtain the image information of the face portion. However, as captured image 100A shown in FIG. 6, in a case where watching target person 50A faces to the side, face detection may not be performed normally in some cases. In the present embodiment, captured image 100A is divided into a plurality of regions 110, and the biological information of watching target person 50A is detected by performing the biological information detection process on each of regions 110. In addition, a region in which the biological information is detected is recognized as person region 115, and the position information of watching target person 50A is detected from a position of person region 115.

The biological information detection device of the present embodiment divides the image data of captured image 100A into the plurality of regions 110 by image region divider 13, and sequentially detects the biological information by biological determiner 14 for each of the divided regions 110. Herein, a detection process of the biological information sequentially performed in units of divided regions is referred to as “vital scan”. In an example of FIG. 6, a process in which the vital scan is performed by scanning in the horizontal direction to the vertical direction from an end portion as a scanning line on the plurality of regions 110 of captured image 100A is shown. In this case, the biological information is detected in a region from the third row from the top and the second row from the left, this region is extracted as person region 115, and it is detected that watching target person 50A normally resides within a range on bed 51 from a positional relationship between person region 115 and bed 51. Biological determiner 14 may start the vital scan from a region in which bed 51 is positioned or a region in the vicinity of bed 51 and may start the vital scan from an end portion of a side close to bed 51, in addition to sequential scanning for an end portion of captured image 100A when scanning the vital scan.

Biological determiner 14 extracts color information in the region, and obtains a representative value of the color information by calculating an average value of, for example, the color information in each of regions 110. Biological determiner 14 detects variation of the representative value of the color information, and determines whether the cycle and state of the variation are close to the motion of the pulse wave of a person. In a case where the variation of the representative value of the color information closes to the pulse wave and variation due to a person is detected, biological determiner 14 estimates that the pulse wave of a person is detected, and determines that the region currently being processed is person region 115 in which a person is present. In addition, biological determiner 14 calculates the pulse rate from the variation of the representative value of the color information of person region 115. Meanwhile, biological determiner 14 determines that it is noise in a case where the variation of the representative value of the color information is different from that of the pulse wave. Biological determiner 14 may detect a region of a color close to a flesh color of a person and determine a person by varying the color of the flesh color region, and perform detection of the biological information, when obtaining the color information of region 110.

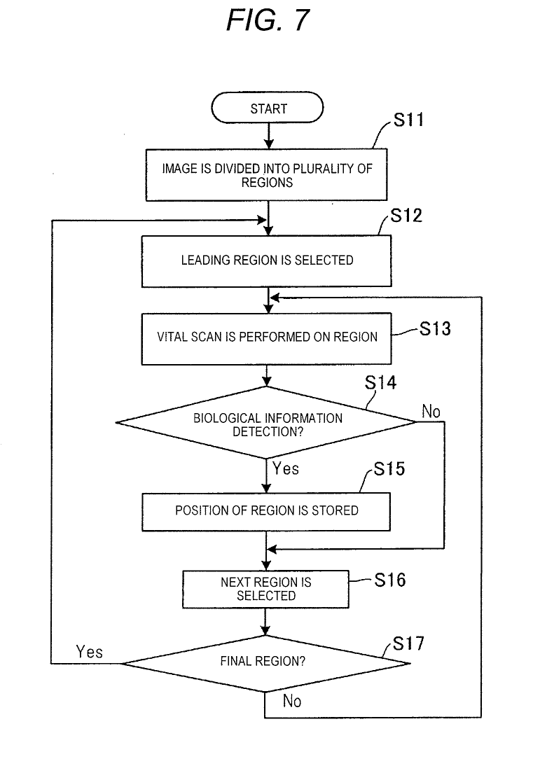

FIG. 7 is a flowchart showing a procedure of the biological information detection process of the first embodiment. Image input 12 inputs the image data of captured image 100A obtained by capture 11. Image region divider 13 divides the image data of captured image 100A into the plurality of regions 110 (S11). Biological determiner 14 selects the leading region 110 at which the vital scan starts (S12), performs the vital scan on the selected region 110 (S13), and determines whether or not the biological information is detected (S14). In a case where the biological information is detected (S14, Yes), biological information and position storage 15 stores the biological information including the pulse rate obtained by biological determiner 14, and stores the position information of person region 115 in which the biological information is detected based on the position information of each region 110 output from image region divider 13 (S15). Next, biological determiner 14 selects the next region 110 in which the vital scan is performed (S16), and it is determined whether there is no next region and the region where the previous vital scan is performed is the final region (S17). In addition, in a case where the biological information is not detected (S14, No), biological determiner 14 selects next region 110 as it is (S16), and determines whether or not it is the final region (S17). In a case where it is not the final region (S17, No), biological determiner 14 performs the vital scan with respect to the selected next region 110 (S13), and repeats the same processes (S13 to S17). In a case where it is the final region (S17, Yes), the process of biological determiner 14 returns to step S12, performs the vital scan again from leading region 110 of the image data of captured image 100A, and repeats the same processes (S12 to S17). When stopping or terminating the biological information detection process, biological determiner 14 receives, for example, a process stop instruction based on an operation instruction from terminal 60 used by watcher 70 or an instruction or the like based on scheduling control of server 30 or the like, and stops or terminates the biological information detection process at an arbitrary timing by interruption of the process stop instruction.

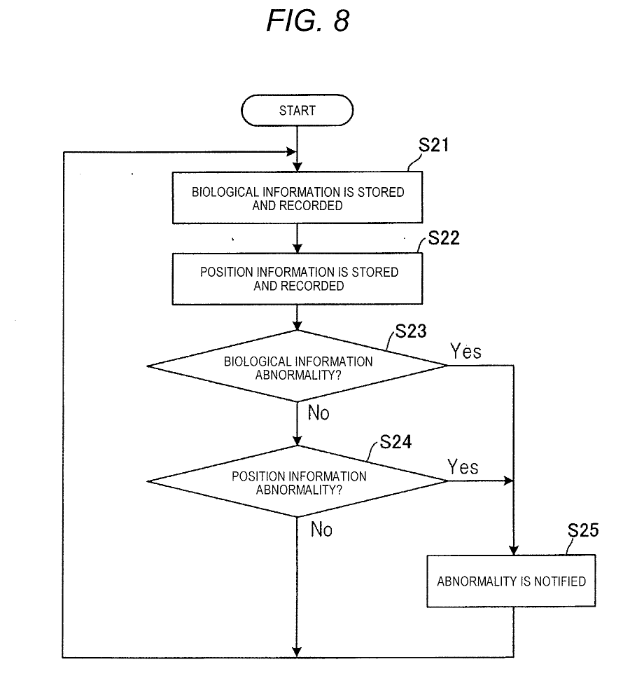

FIG. 8 is a flowchart showing a procedure of an abnormal state detection process in the present embodiment. Information input 31 obtains the biological information of watching target person 50A including the pulse rate detected from captured image 100A, and records the obtained information in recorder 32 (S21). In addition, information input 31 obtains the position information of watching target person 50A detected from captured image 100A, and records the obtained information in recorder 32 (S22). Biological information abnormality detector 33 refers the obtained biological information of watching target person 50A, and determines whether or not there is an abnormality in the biological information (S23). For example, biological information abnormality detector 33 determines the presence or absence of the abnormal state of the biological information such as sharp increase, decrease, and stop of the pulse rate. Position information abnormality detector 34 refers the obtained position information of watching target person 50A, and determines whether or not there is an abnormality in the position information (S24). For example, when watching target person 50A resides at a position other than the bed for a predetermined time or more, position information abnormality detector 34 determines the presence or absence of the abnormal state of the position information. In a case where there is an abnormality in the biological information or there is an abnormality in the position information, abnormality notifier 36 transmits the abnormality notification to terminal 60 used by watcher 70, and notifies the occurrence of the abnormal state (S25).

For example, in a case where terminal 60 includes a display, when the abnormality notification is received from server 30, abnormality notification information is displayed on a display. For example, in a case where terminal 60 includes a lamp such as an LED, when the abnormality notification is received from server 30, the occurrence of abnormality is notified by turning on of the lamp. For example, in the case of including a speaker or a buzzer, when receiving the abnormality notification from server 30, an audio is output from the speaker or the buzzer of terminal 60. Terminal 60 may receive the biological information from server 30 for each predetermined time or at an arbitrary timing, and may display the biological information of watching target person 50.

According to the present embodiment, since the face is turned against the camera as the watching target person as the observed person becomes sideways or lying down, even in a case where the face of the watching target person cannot be detected, it is possible to accurately detect the biological information of the watching target person by detecting the biological information in units of a plurality of divided regions. In addition, it is possible to obtain the position information of the watching target person by the region in which the biological information is detected. In addition, by detecting and notifying the abnormality of the biological information or the position information which is obtained, a watcher or the like who is outside the room can appropriately grasp the abnormal state of the watching target person.

As described above, camera 10A of an example of the biological information detection device of the present embodiment includes image input 12 for receiving the image data captured by the observed person, image region divider 13 for dividing the image data into a plurality of regions, biological determiner 14 for determining the presence or absence of the detection of the biological information to be estimated as the pulse wave of a person in each of regions, and biological information and position storage 15 for obtaining the detected biological information, obtaining the position information of the region in which the biological information is detected, and storing the biological information and the position information.

With this, even in a case where the face of the observed person cannot be detected, it is possible to accurately detect the biological information of the observed person by the biological information detection in each of the plurality of divided regions. In addition, by a position of the region in which the biological information is detected, it is possible to obtain the position information of the observed person, and it is possible to grasp the position of the observed person.

In addition, camera 10A determines the presence or absence of the detection of the biological information in each region by sequentially performing the biological information detection process with respect to the plurality of regions in biological determiner 14. With this, by sequentially performing the biological information detection process in the plurality of the divided regions, even in a case where the face of the observed person cannot be detected, it is possible to determine whether or not the biological information by determination for each region is detected.

In addition, the biological information detection system of the present embodiment is a system including camera 10A, as an example of the biological information detection device, for detecting the biological information based on the image data captured by the observed person, and server 30, as the biological information processing device, for processing the biological information obtained by the biological information detection device. Camera 10A receives the image data captured by the observed person, divides the image data into the plurality of regions, determines the presence or absence of the detection of the biological information to be estimated as the pulse wave of a person in each of regions, obtains the detected biological information, obtains the position information in the region in which the biological information is detected, and outputs the biological information and the position information to server 30. Server 30 receives the biological information and the position information, detects the abnormality of the received biological information, detects the abnormality of the input position information, and notifies the abnormality in a case where at least one abnormal state in the biological information and the position information is detected.

With this, even in a case where the face of the observed person cannot be detected, by the biological information detection in each of the plurality of divided regions, it is possible to accurately detect the biological information of the observed person. In addition, by the position of the region in which the biological information is detected, it is possible to obtain the position information of the observed person, and it is possible to grasp the position of the observed person. In addition, by detecting the abnormality of the biological information and the position information and performing the abnormality notification, in the watcher or the like who watches the observed person, it is possible to detect the occurrence of the abnormal state relating to the observed person, and it is possible to accurately perform correspondence at the time of the abnormality.

### Second Embodiment

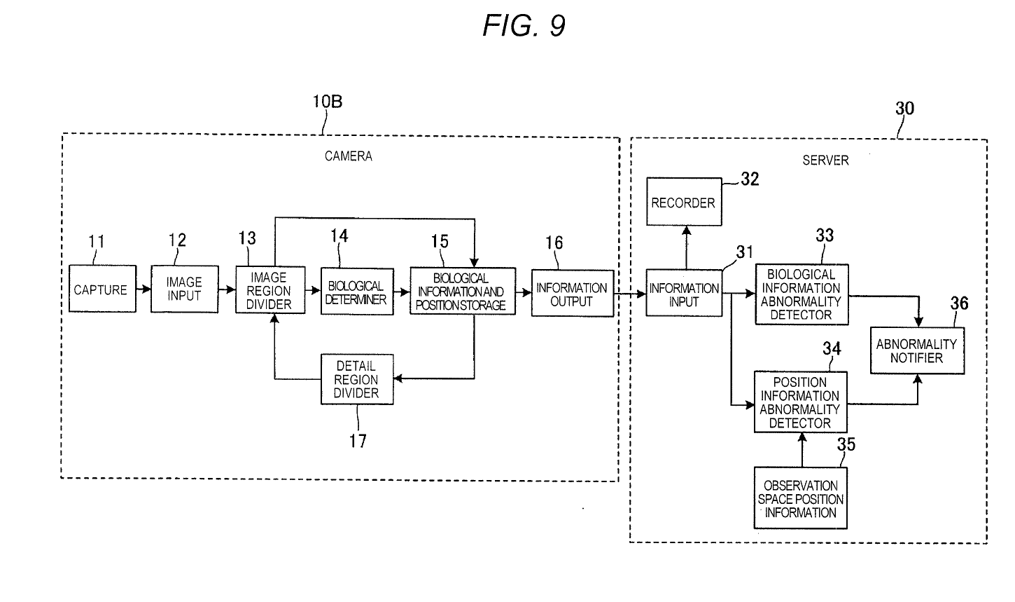

FIG. 9 is a block diagram showing a functional configuration of the biological information detection system of a second embodiment. In the second embodiment, a processing example for further subdividing the regions when detecting the biological information by dividing the captured image of the monitoring target into the plurality of regions is shown. Camera 10B of the second embodiment is configured to include capture 11, image input 12, image region divider 13, biological determiner 14, biological information and position storage 15, detail region divider 17, and information output 16. Here, the constituent elements different from those of the biological information detection system of the first embodiment shown in FIG. 2 will be mainly described, the same constituent elements will be denoted by the same reference numerals, and description thereof will be omitted.

Detail region divider 17 divides the region into a plurality of detail regions by further subdividing the region with respect to person region 115 in which the biological information of watching target person 50 is detected, by biological determiner 14. For example, detail region divider 17 divides the image data into grid type detail regions of a plurality of sections by dividing the image data into a plurality of image data in each of the vertical direction and the horizontal direction of the image, in person region 115 which are divided into the plurality of regions and in which the biological information is detected. Biological determiner 14 sequentially performs the detection of the biological information in units of detail regions subdivided by detail region divider 17. With this, it is possible to obtain highly accurate biological information and the position information. As a subdividing process on a region by detail region divider 17, in addition to a process of finely dividing the region in which the biological information is detected, a process for fining a size of a detection target region of the biological information may be used by changing or the like an angle of view of the captured image by panning, zooming, or the like of capture 11 of camera 10B.

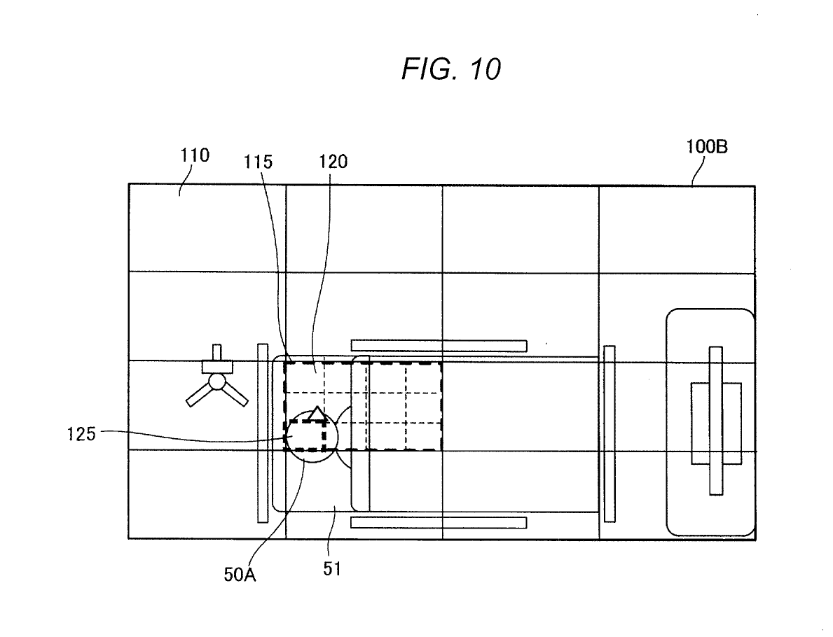

Next, each process of the detection of the biological information and the detection of the abnormal state in the second embodiment will be described. FIG. 10 is a diagram for explaining a biological information detection process in the second embodiment. In the present embodiment, captured image 100B is divided into the plurality of regions 110, the biological information of watching target person 50A is detected by performing the biological information detection process in each of regions 110, person region 115 in which the biological information is further detected is divided into a plurality of detail regions 120, and the biological information is detected in each of detail regions 120. In addition, the position information of watching target person 50A is detected from person region 125 in which the biological information is detected among the plurality of detail regions 120.

The vital scan in the present embodiment is also performed in detail region 120 as well as region 110 before the subdivision. In an example of FIG. 10, in person region 115 of captured image 100B in which the biological information is detected, a process for performing the vital scan by scanning the plurality of detail regions 120 from an end portion in the vertical direction and horizontal direction as the scanning line is shown. In this case, the biological information is detected in a detail region from the third row from the top and the first row from the left, this detail region is extracted as person region 125, and it is detected that watching target person 50A normally resides within a range on bed 51 from a positional relationship between person region 125 and bed 51. Biological determiner 14 detects the variation of the representative value of the color information and determines whether or not the cycle and mode of the variation are close to the motion of the pulse wave of a person in each detail region 120. In a case where the variation of the representative value of the color information is close to the pulse wave and variation of a person is detected, biological determiner 14 estimates that the pulse wave of a person is detected and determines that the detail region being processed currently is person region 125 in which the person resides. In addition, biological determiner 14 calculates the pulse rate from the variation of the representative value of the color information of person region 125. Meanwhile, in a case where the variation of the representative value of the color information is different from that of the pulse wave, biological determiner 14 determines that it is noise.

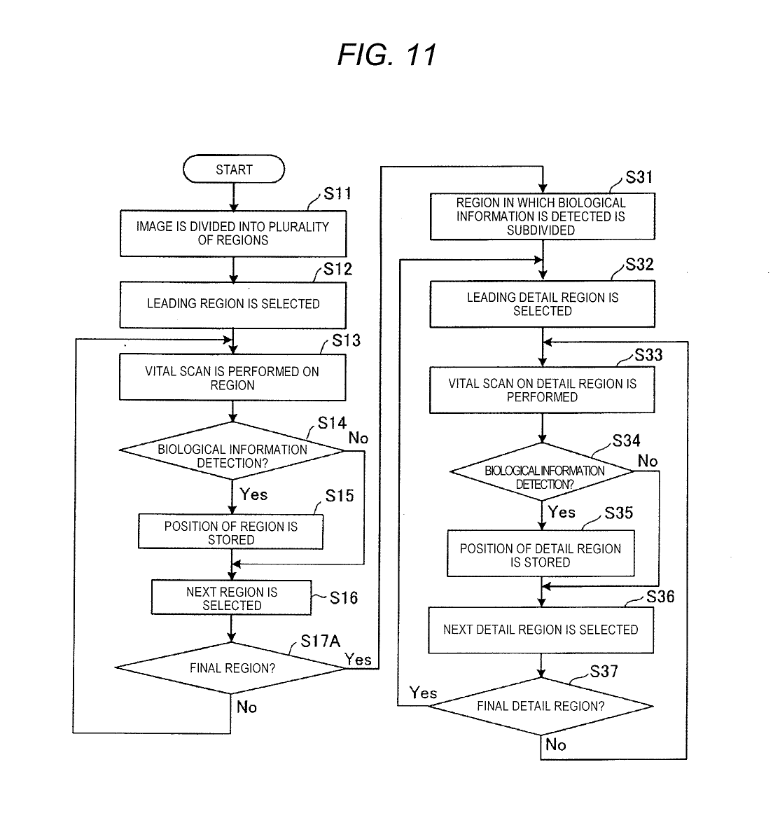

FIG. 11 is a flowchart showing a procedure of the biological information detection process of the second embodiment. Image input 12 inputs the image data of captured image 100B obtained by capture 11. Processes of steps S11 to S16 by image region divider 13 and biological determiner 14 are the same as those of the first embodiment shown in FIG. 7. Biological determiner 14 performs the detection process of the biological information in units of the divided region, and determines whether or not the current processing target region is the final region (517A). In a case where it is the final region (517A, Yes), detail region divider 17 subdivides the region so as to further divide person region 115 in which the biological information is detected into the plurality of detail regions 120 (S31).

Biological determiner 14 selects leading detail region 120 in which the vital scan starts (S32), performs the vital scan on the selected detail region 120 (S33), and determines whether or not the biological information is detected (S34). In a case where the biological information is detected (S34, Yes), biological information and position storage 15 stores the biological information including the pulse rate including the pulse rate obtained by biological determiner 14, and stores the position information of person region 125 in which the biological information is detected based on the position information in each of detail regions 120 output from image region divider 13 (S35). Next, biological determiner 14 selects next detail region 120 on which the vital scan is performed (S36), and determines whether there is no next detail region and the region where the previous vital scan is performed is the final detail region (S37). In addition, in a case where the biological information is not detected (S34, No), biological determiner 14 selects next detail region 120 as it is (S36), and determines whether or not it is the final detail region (S37). In a case where it is not final detail region (S37, No), biological determiner 14 performs the vital scan (S23) and repeats the same processes (S33 to S37) with respect to the selected next detail region 120. In a case where it is the final detail region (S37, Yes), the process of biological determiner 14 returns to step S32, performs the vital scan again from leading detail region 120, and repeats the same processes (S32 to S37) on person region 115 in which the biological information is detected. In a case where the biological information detection process is stopped or terminated, biological determiner 14 receives a process stop instruction based on an operation instruction from terminal 60 used, for example, by watcher 70, an instruction by scheduling control of server 30, or the like, stops or terminates a biological information detection process at an arbitrary timing by interruption of the process stop instruction.

According to the present embodiment, by performing the biological information detection in units of the plurality of divided region and the biological information detection on the detail region where the region in which the biological information is detected is further subdivided, since a feature amount for each region is more remarkably obtained in the detail region, it is possible to obtain highly accurate biological information from a region having a lot of skin color such as a face region of the watching target person.

As described above, camera 10B as an example of the biological information detection device of the present embodiment includes only detail region divider 17 obtained by further subdividing the region in which the biological information is detected by image region divider 13, and biological determiner 14 determines the presence or absence of the detection of the biological information by performing the biological information detection process on the subdivided detail region. With this, by performing the detection of the biological information on the subdivided detail region, since, for example, there is a high possibility that biological information of a region having a large amount of component of the feature amount of a person such as the region having a lot of skin color can be obtained, it is possible to obtain the highly accurate biological information.

### Third Embodiment

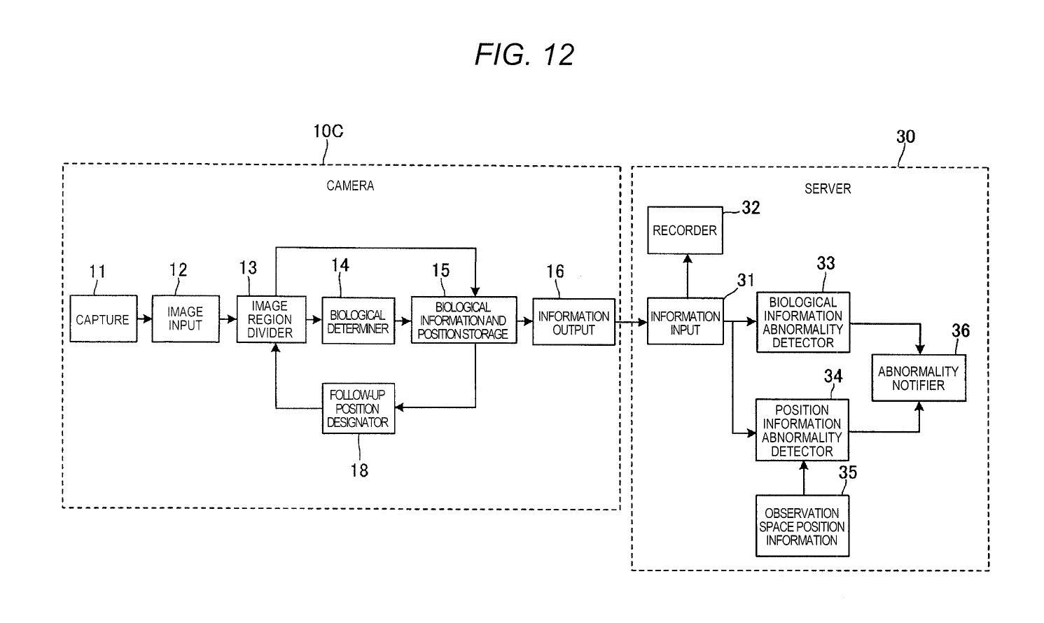

FIG. 12 is a block diagram showing a functional configuration of a biological information detection system of a third embodiment. In the third embodiment, a process example for causing a person region to follow the motion of the watching target person in the detection of the biological information after the next time on the person region in which the biological information to be estimated that the watching target person resides is detected will be described. Camera 10C of the third embodiment is configured to include capture 11, image input 12, image region divider 13, biological determiner 14, biological information and position storage 15, follow-up position designator 18, and information output 16. Here, the constituent elements different from those of the biological information detection system of the first embodiment shown in FIG. 2 will be mainly described, the same constituent elements will be denoted by the same reference numerals, and description thereof will be omitted.

Follow-up position designator 18 detects motion of person region 115 in which the biological information is detected by biological determiner 14 in the captured image, and causes person region 115 to be followed according to the motion of the watching target person by designating the position of person region 115 after movement by calculating a movement amount of person region 115. Follow-up position designator 18 detects feature amount of the image data of person region 115, determines whether or not there is a motion of the feature amount based on position change of the feature amount, and calculates the movement amount of person region 115. For example, as the feature amount of the image data, a skin color region may be used. Follow-up position designator 18 causes person region 115 to be moved and followed according to the calculated movement amount of person region 115. With this, in order to cause the motion of the watching target person to be followed in a unit of person region 115 in which the biological information is detected, it is possible to continuously monitor the watching target person.

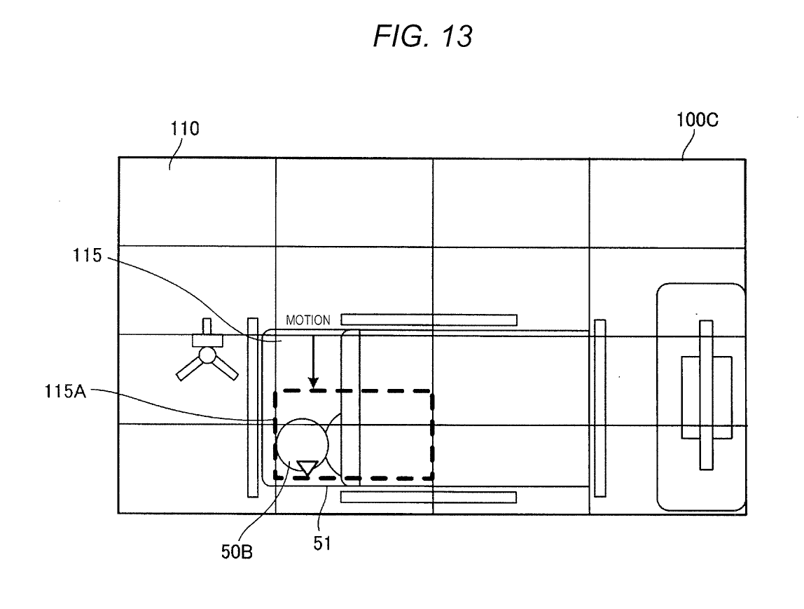

Next, each process of detection of the biological information and the detection of the abnormal state in the third embodiment will be described. FIG. 13 is a diagram for explaining the biological information detection process in a first example of the third embodiment. In the present embodiment, in a case where the biological information of watching target person 50B is detected and further watching target person 50B is moved with respect to person region 115 in which the biological information is detected, by dividing captured image 100C into the plurality of regions 110 and performing the biological information detection process on each of regions 110, the motion of person region 115A is detected and followed. In addition, the position information of watching target person 50B is detected by person region 115A to be followed to the motion. The first example is a processing example of causing person region 115 to be followed by the plurality of divided regions 110 represented in the first embodiment.

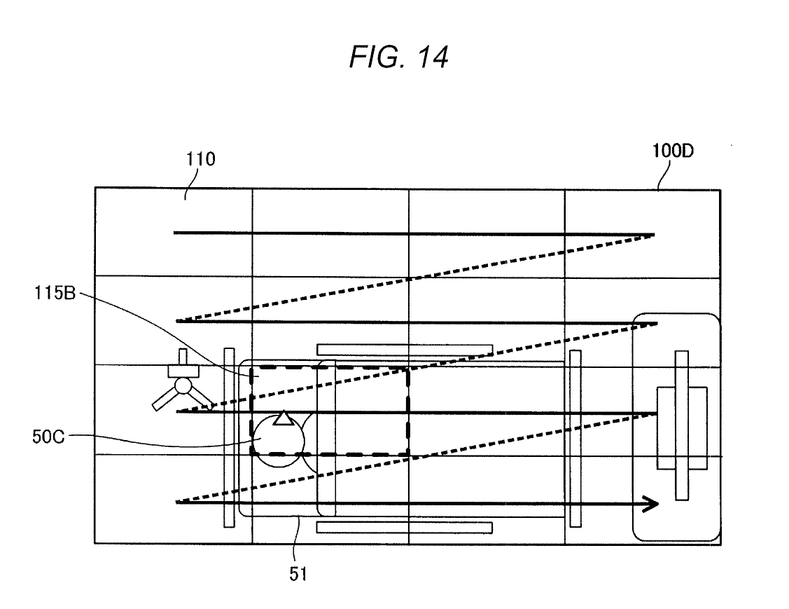

In addition, FIG. 14 is a diagram for explaining a re-detection process in a case where the biological information cannot be detected. In a case where the biological information cannot be detected in person region 115A to be followed by the motion of the feature amount in a screen of captured image 100D, the vital scan is performed again on the entirety of captured image 100D and the biological information is sequentially detected for each of the plurality of divided regions 110. With this, for example, as watching target person 50B is lying down, turning and moving greatly, even in a case where the following of person region 115A is stopped due to the movement of watching target person 50B and biological information cannot be detected. It is possible to detect the biological information by performing the vital scan on the entire image again.

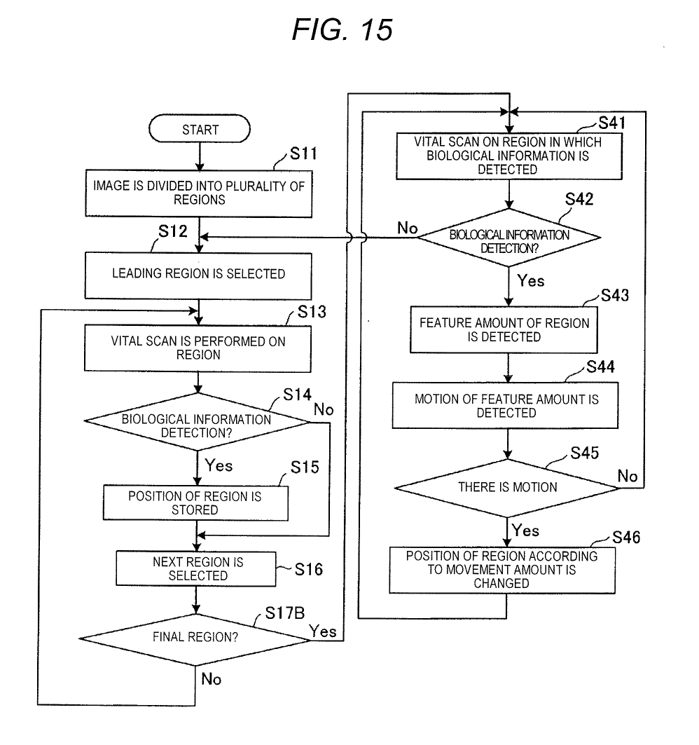

FIG. 15 is a flowchart showing a procedure of the biological information detection process of the first example of the third embodiment. Image input 12 inputs the image data of captured image 100C obtained by capture 11. Processes of steps S11 to S16 by image region divider 13 and biological determiner 14 are the same as those of the first embodiment shown in FIG. 7. Biological determiner 14 performs the detection process of the biological information in units of the divided region, and determines whether or not the current processing target region is the final region (S17B). In a case where it is the final region (S17B, Yes), biological determiner 14 performs the vital scan on person region 115 in which the biological information is detected (S41), and determines whether or not the biological information is detected (S42). In a case where the biological information is detected (S42, Yes), follow-up position designator 18 detects the feature amount of the image data of person region 115 (S43). In addition, follow-up position designator 18 detects the motion of the feature amount by the position change of the feature amount of the image data (S44). Follow-up position designator 18 determines whether or not there is the motion of person region 115 by the movement amount of the feature amount (S45). In a case where the motion of the feature amount is detected (S45, Yes), follow-up position designator 18 changes the position of person region 115A in accordance with the movement amount of the feature amount, and causes person region 115A to be followed (S46). The process of biological determiner 14 returns to step S41, and biological determiner 14 performs the vital scan on person region 115 after following, and repeats the same processes (S41 to S45). Meanwhile, in a case where the motion of the feature amount is not detected by follow-up position designator 18 (S45. No), the process returns to step S41, and biological determiner 14 performs again the vital scan on person region 115, and repeats the same processes (S41 to S45). In a case where the biological information detection process is stopped or terminated, biological determiner 14 receives the process stop instruction based on, for example, the operation instruction from terminal 60 used by watcher 70, an instruction by the scheduling control of server 30, or the like, and stops or terminates the biological information detection process at an arbitrary timing by interrupt of the process stop instruction.

In addition, in a case where the biological information is not detected (S42, No), the process of biological determiner 14 returns to step S12, as shown in FIG. 14, the vital scan is performed again from leading region 110 of the image data of captured image 100D, and the same processes are repeated (S12 to S17B). With this, person region 115B where watching target person 50C resides is detected by performing the re-detection process of the biological information.

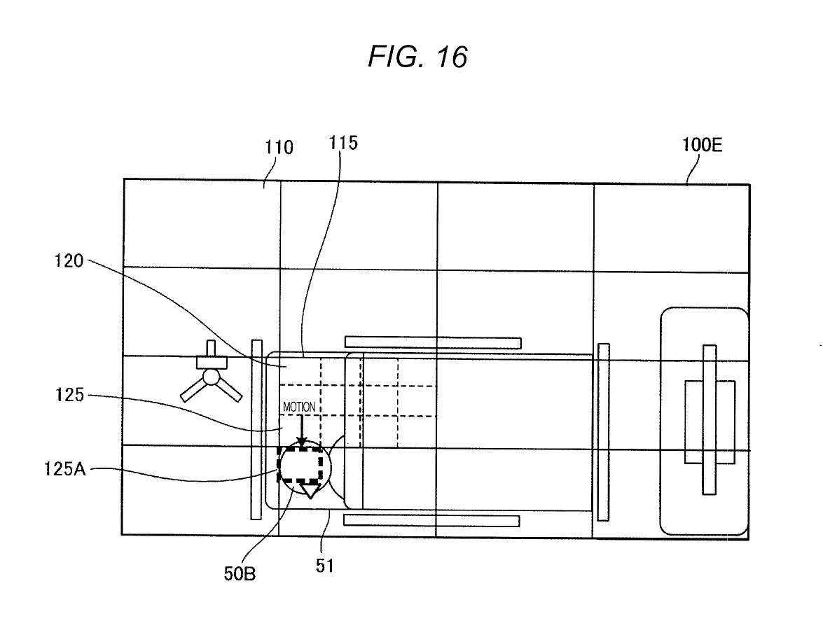

FIG. 16 is a diagram for explaining the biological information detection process in a second example of the third embodiment. The second example is a process example to cause person region 125 to be followed by detail region 120 represented in the second embodiment. In the second example, the biological information detection process is performed on region 110 in which captured image 100E is divided into a plurality of regions, the biological information of watching target person 50B is further detected by performing the biological information detection process on detail region 120 divided into a plurality of regions on person region 115 in which the biological information is detected, and the motion of person region 125A is detected and followed with respect to person region 125 in which the biological information is detected in a case where watching target person 50B is moved. In addition, the position information of watching target person 50B is detected by person region 125A following the motion.

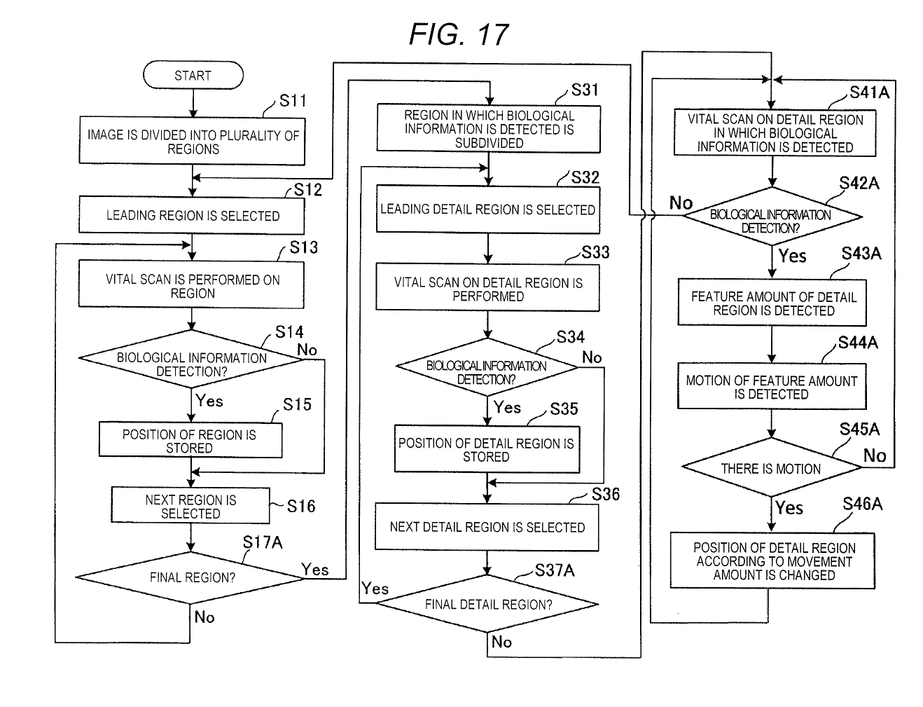

FIG. 17 is a flowchart showing a procedure of a biological information detection process of the second example of the third embodiment. Image input 12 inputs the image data of captured image 100E obtained by capture 11. Processes of steps S11 to S16 by image region divider 13 and biological determiner 14 are the same as those of the first embodiment shown in FIG. 7. In addition, processes of step S17A and S31 to S36 by image region divider 13 and biological determiner 14 are the same as those of the second embodiment shown in FIG. 11. Biological determiner 14 performs the detection process of the biological information in units of the divided detail region, and determines whether or not the current processing target region is the final detail region (S37A). In a case where it is the final region (S37A, Yes), biological determiner 14 performs the vital scan on person region 125 of the detail region in which the biological information is detected (S41A), and determines whether or not the biological information is detected (S42A).

In a case where the biological information is detected (S42A, Yes), follow-up position designator 18 detects the feature amount of the image data in person region 125 of the detail region (S43A). In addition, follow-up position designator 18 detects the motion of the feature amount by position change of the feature amount of the image data (S44A). Follow-up position designator 18 determines whether or not there is the motion of person region 125 by the movement amount of the feature amount (S45A). In a case where the motion of the feature amount is detected (S45A, Yes), follow-up position designator 18 changes the position of person region 125A of the detail region in accordance with the movement amount of the feature amount, and causes person region 125A to be followed (S46A). The process of biological determiner 14 returns to step S41A, the vital scan is performed on person region 125 after the following, and the same processes are repeated (S41A to S45A).

Meanwhile, in a case where the motion of the feature amount is not detected by follow-up position designator 18 (S45A, No), the process returns to step S41A, biological determiner 14 performs again the vital scan on person region 125 of the detail region, and the same processes are repeated (S41A to S45A). In a case where the biological information detection process is stopped or terminated, biological determiner 14 receives the process stop instruction based on, for example, the operation instruction from terminal 60 used by watcher 70, an instruction by the scheduling control of server 30, or the like, and stops or terminates the biological information detection process at an arbitrary timing by interrupt of the process stop instruction.

According to the present embodiment, by causing a detection region of the biological information to be followed, it is possible to continue the detection of the biological information adapting to the movement even in a case where the watching target person is moved, and it is possible to obtain the biological information without missing the biological information of the watching target person. In addition, it is possible to obtain the position information of the watching target person following the movement, and it is possible to obtain highly accurate position information.

As described above, camera 10C as an example of the biological information detection device of the present embodiment includes follow-up position designator 18 for causing the position of the region in which the biological information in the image data is detected, to be followed according to the motion of the region, and biological determiner 14 determines the presence or absence of the detection of the biological information by performing the biological information detection process on the region after the following. With this, even in a case where there is the motion of the observed person, it is possible to continue the detection of the biological information in accordance with the motion, and it is possible to reduce omission of detection of the biological information.

So far, while various embodiments have been described with reference to the drawings, it goes without saying that the present disclosure is not limited to such examples. Those skilled in the art will appreciate that various modifications or modifications can be conceived within the scope described in the claims and they also naturally fall within the technical scope of the present disclosure. In addition, the constituent elements in the above embodiment may be arbitrarily combined without departing from the gist of the embodiment.

The present disclosure is useful as the biological information detection device, the biological information detection method, and the biological information detection system which accurately detect the biological information of the observed person in a case where the face of the observed person cannot be detected.

1. A biological information detection device comprising:
an image input that inputs image data captured by an observed person;
an image region divider that divides the image data into a plurality of regions;
a biological determiner that determines presence or absence of detection of biological information to be estimated as a pulse wave of a person in each of the regions;
a biological information obtainer that obtains the detected biological information; and
a position information obtainer that obtains position information of a region in which the biological information is detected.

2. The biological information detection device of claim 1,
wherein the biological determiner determines the presence or absence of the detection of the biological information in each region by sequentially performing a biological information detection process on the plurality of regions.

3. The biological information detection device of claim 1,
wherein the image region divider includes a detail region divider that further subdivides the region in which the biological information is detected, and
wherein the biological determiner determines the presence or absence of the detection of the biological information by performing the biological information detection process on the subdivided detail region.

4. The biological information detection device of claim 1, further comprising:
a follow-up position designator that causes a position of the region in which the biological information in the image data is detected to be followed according to motion of the region,
wherein the biological determiner determines the presence or absence of the detection of the biological information by performing the biological information detection process on the region after following-up.

5. A biological information detection method in a biological information detection device, the method comprising:
receiving image data captured by an observed person;
dividing the image data into a plurality of regions;
determining presence or absence of detection of biological information to be estimated as a pulse wave of a person in each of the regions;
obtaining the detected biological information; and
obtaining position information of a region in which the biological information is detected.

6. A biological information detection system comprising:
a biological information detection device that detects biological information based on image data captured by an observed person; and
a biological information processing device that processes the biological information obtained by the biological information detection device,
wherein the biological information detection device
receives the image data captured by the observed person,
divides the image data into a plurality of regions,
determines presence or absence of detection of the biological information to be estimated as a pulse wave of a person in each of the regions,
obtains the detected biological information,
obtains position information of a region in which the biological information is detected, and
outputs the biological information and the position information to the biological information processing device, and
wherein the biological information processing device
receives the biological information and the position information,
detects abnormality of the received biological information,
detects abnormality of the received position information, and
notifies the abnormality in a case where an abnormal state of at least one of the biological information and the position information is detected.
